(19) United States
(12) Patent Application Publication
(10) Pub. No.: US 2006/0067938 A1
(43) Pub. Date: Mar. 30, 2006

(54) Methods for the treatment of osteoarthritis and compositions thereof

(76) Inventors: Sherif Daouti, Hasbrouck Heights, NJ (US); Chanrika S. Kumar, Maynard, MA (US); Brian J. Latario, Groton, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/491,798

(22) PCT Filed: Oct. 25, 2002
(86) PCT No.: PCT/EP02/11955

§ 371 Date: Apr. 4, 2005

Related U.S. Application Data
(60) Provisional application No. 60/343,716, filed on Oct. 26, 2001.

(51) Int. Cl. A61K 31/5377; A61K 39/395

(52) U.S. Cl. 424/146.1; 514/232.5

(57) ABSTRACT

The invention discloses PI3K, PKB, IL-1, and OSM as suitable targets for the development of new therapeutics to treat, prevent or ameliorate osteoarthritis (OA). The invention relates to methods to treat, prevent or ameliorate OA and pharmaceutical compositions therefor comprising substances with inhibitory effects on PI3K and/or PKB enzyme activity and/or gene expression and IL-1 and/or OSM activity or gene expression. The invention also relates to a method to identify compounds with therapeutic usefulness to treat OA, comprising identifying compounds that can effect PI3K and/or PKB activity and/or gene expression and/or IL-1 or OSM activation of PI3K and/or PKB activity and/or gene expression which leads to downregulation of AGG-1 and/or COL-3 and thus prevent, treat or ameliorate OA in vivo.

### BACKGROUND OF THE INVENTION

Arthritis, is defined as a general inflammation of articular structures (joints) in the body. Rheumatoid arthritis, is a subset of arthrititic diseases, which is a chronic systemic disease that may be caused by autoimmune mechanisms and/or viral infections and involves inflammation of synovial membranes and articular structures. It is usually polyarticular in nature. In contrast to general arthritis and rheumatoid arthritis, the disease state of osteoarthritis (OA) is a complex, multi-factorial progressive disease that is non-inflammatory in nature and which is characterized by a general age-related degradation of articular cartilage in the joints. OA is also characterized by chondrocyte activation leading to cell proliferation and apoptosis, protease expression and abnormal matrix production, failed cartilage repair leading to loss of extracellular matrix, matrix calcification and osteophyte formation. The degradation of cartilage and extracellular matrix structures leads to increased friction between the bones and nerves of the affected joints. OA causes varying levels of pain and progressive debilitation in those afflicted with the disease. Current therapies for OA are pallative or surgical.

While the specific molecular pathways involved in the onset of the degradative processes seen in OA are not known, it is postulated that a central mechanism leading to cartilage loss is the enzymatic degradation of type II collagen and aggrecan by the collagen degrading enzymes aggrecanase-1 (AGG-1) aggrecanase-II (AGG-2) and collagenase-3 (COL-3) which are produced by articular chondrocytes. While COL-3 appears to be the most efficient collagenase for type II collagen and aggrecan cleavage is predominantly at AGG-1 and AGG-2 cleavage sites, the role of specific aggrecanases in OA and mechanisms that lead to the induction of these enzymes have yet to be determined.

Expression and production of the matrix degrading metalloproteinase, COL-3, in chondrocytes, can be enhanced by platelet derived growth factor (PDGF). Protein Kinase B (PKB) is a proto-oncogene which is activated by growth factors, cytokines and cellular stress conditions. PDGF is a potent activator of PKB and also of Phospho-Inosotide 3 Kinase (PI3K) which is an upstream activator of PKB. However, the mechanisms by which PDGF activates matrix metalloproteinases and the relationship between PI3K, PKB and OA have not been elucidated.

We have now surprisingly discovered that PI3K and PKB in adition to cytokines such as interleukin-1 (IL-1) and oncostatin M (OSM) play a role in cartilage loss associated with OA. IL-1, OSM and PDGF are are shown to significantly up-regulate matrix metalloproteinase gene expression via the PI3K and PKB pathways. Thus the present invention contemplates the use of modulators and inhibitors of IL-1, OSM, PI3K, and/or PKB to reduce activation of the PI3K/PKB signalling pathway which will in turn reduce induction of AGG-1 and COL-3 gene expression. Given the destructive role of AGG-1 and COL-3 in the pathogenesis of OA, PI3K, PKB, IL-1, and OSM and isoforms thereof may be used as novel drug targets for OA or for any diseases associated with altered levels of AGG-1 or COL-3. The invention also provides methods for identifying modulators and inhibitors of PI3K, PKB, IL-1 and OSM induced AGG-1 and COL-3 activity and also for identifying modulators of PI3K, PKB, IL-1, and OSM gene expression and the use of such modulators for the treatment and/or prevention of OA in subjects. The invention also provides pharmaceutical compositions of said modulators and uses for said pharmaceutical compositions.

### SUMMARY OF THE INVENTION

The invention is based on the inventors' novel discovery that in vitro, and in vivo, PI3K, PKB, IL-1, and OSM lead to increases in the induction of AGG-1 and COL-3 mRNA. The inventors have for the first time produced evidence for increased AGG-1 and COL-3 mRNA induction due to PI3K, PKB, IL-1, and OSM. This finding is shown by the use of the PI3K and PKB inhibitor, LY294002, and an inhibitory dominant mutant form of PKB. Levels of AGG-1 and COL-3 mRNA activation in human articular chondrocytes, due to the PI3K and PKB activator PDGF, were reduced in the presence of LY294002 and the dominant inhibitory mutant of PKB. In addition, both IL-1 and OSM induced higher levels of AGG-1 and COL-3 mRNA induction than did PDGF. This IL-1 and OSM “superinduction” resulted in increased levels of AGG-1 and COL-3 mRNA compared to non-IL-1 and OSM induced chondrocytes. Further, the superinduction is maintained, though to an attenuated level, even in the presence of LY294002 and the dominant inhibitory mutant. The inventors thus show that modulators or inhibitors of PI3K, PKB, IL-1, and OSM may be useful for the treatment of OA via a reduction of AGG-1 and COL-3 expression and production. Given the destructive role of AGG-1 and COL-3 in the pathogenesis of OA, PI3K, PKB, IL-1, and OSM and isoforms thereof may be used as novel drug targets for OA. Thus, it is contemplated herein that PI3K, PKB, IL-1 and OSM and isoforms thereof are useful drug targets for the development of therapeutics to treat, prevent or ameliorate OA, a disease state not previously known to involve the induction of AGG-1 and COL-3 by PI3K and PKB activation and IL-1 and OSM induced PI3K and PKB activation.

Thus, in one aspect the invention provides a method for identifying modulators useful for the treatment, prevention or amelioration of osteoarthritis comprising testing whether candidate compounds are capable of altering or inhibiting the enzymatic activity of PI3K and/or PKB or altering or inhibiting PI3K, PKB, IL-1, and/or OSM gene expression in vitro or in vivo and thereby downregulating gene expression of AGG-1 or COL-3 production in chondrocytes, wherein altered expression of AGG-1 or COL-3 protein indicates a compound may have potential therapeutic value.

In one embodiment, the present invention provides a method to prevent, treat, or ameliorate a disease associated with or caused by altered levels of AGG-1 or COL-3 comprising administering to a subject in need thereof an effective amount of a compound capable of modulating PI3K, PKB, IL-1, or OSM and through said modulation alter expression of AGG-1 or COL-3.

Another aspect of the invention relates to a method to prevent, treat, or ameliorate osteoarthritis comprising administering to a subject in need thereof an effective amount of a compound capable of modulating PI3K, PKB, IL-1, or OSM and through said modulation alter expression of AGG-1 or COL-3.

Yet another aspect of the invention relates to the method to prevent, treat, or ameliorate osteoarthritis comprising administering to a subject in need thereof an effective amount of a compound capable of modulating PI3K, PKB, IL-1, or OSM and through said modulation alter expression of AGG-1 or COL-3 wherein said PI3K modulato, PKB modulator, IL-1 modulator, or OSM modulator inhibits in said subject the enzyme activity of one or more proteins selected from the following: PI3K and PKB.

In another embodiment, the invention relates to the method to prevent, treat, or ameliorate osteoarthritis comprising administering to a subject in need thereof an effective amount of a compound capable of modulating PI3K, PKB, IL-1, or OSM and through said modulation alter expression of AGG-1 or COL-3 wherein said PI3K modulator, PKB modulator, IL-1 modulator, or OSM modulator inhibits in said subject gene or protein expression of one or more proteins selected from the following: PI3K, PKB, IL-1, OSM, AGG-1, and COL-3.

A known PI3K and/or PKB inhibitor, includes a compound referred to herein as LY-290042 (also known as 2-(4-morpholinyl)-8-phenyl-4H-1-benzopyran-4-one), (Vlahos, C J, et al. J. Biol. Chem. (1994) 269: 7 5241-5248) and commercially available from Sigma-Aldrich, St. Louis, Mo. Based on the surprising good results observed as the reduction of levels of AGG-1 and COL-3, it is contemplated herein that a pharmaceutical composition comprising this compound may be used to inhibit PI3K and/or PKB and through this inhibition reduce AGG-1 and COL-3 mRNA and protein levels in chondrocytes and thus can be useful to treat, prevent or ameliorate OA in a subject in need thereof.

The discovery that a PI3K and/or PKB inhibitor, LY249002 2-(4-morpholinyl)-8-phenyl-4H-1-benzopyran-4-one), alters expression of AGG-1 or Collagenease-3 is novel and constitutes a key aspect of the invention. Therefore, in one embodiment, the invention relates to a method to prevent, treat, or ameliorate osteoarthritis comprising administering to a subject in need thereof an effective amount of a compound capable of modulating PI3K or PKB and through said modulation alter expression of AGG-1 or COL-3 wherein said PI3K modulator or PKB modulator is LY249002 2-(4-morpholinyl)-8-phenyl-4H-1-benzopyran-4-one) in free or pharmaceutically acceptable salt forms.

Another aspect of the invention relates to the method of claim 2 wherein said PI3K modulator, PKB modulator, IL-1 modulator, or OSM modulator comprises any one or more substances selected from the following: antisense oligonucleotides, triple helix DNA, single stranded DNA, ribozymes, RNA aptamer and double stranded RNA wherein said substances are designed to inhibit gene expression of PI3K, PKB, IL-1, OSM, AGG-1 or COL-3 proteins.

A further aspect of the invention relates to the use a compound capable of modulating Phospho-Inositide 3 Kinase or Protein Kinase B for the manufacture of a medicament for the treatment of a disease associated with or caused by altered levels of Aggrecanase-1 or Collagenase-3. In a preferred embodiment the compound is capable of inhibiting Phospho-Inositide 3 Kinase or Protein Kinase B. In another preferred embodiment, the disease is osteoarthritis. In a particularly preferred embodiment the compound capable of inhibiting Phospho-Inositide 3 Kinase or Protein Kinase B is 2-(4-morpholinyl)-8-phenyl-4H-1-benzopyran-4-one).

An interesting embodiment of the invention relates to pharmaceutical compositions which comprise antibodies that are highly selective for human PI3K, PKB, IL-1, OSM, AGG-1 and/or COL-3 polypeptides or portions of human PI3K, PKB, IL-1, OSM, AGG-1 and/or COL-3 polypeptides. Antibodies to these proteins may cause the aggregation of these proteins in a subject and thus reduce the activity of the enzymes. Such antibodies may also decrease enzymatic activity, for example, by interacting directly with active sites or by blocking access of substrates to active sites. PI3K, PKB, IL-1, OSM, AGG-1 and/or COL-3 antibodies may also be used to inhibit enzymatic activity of these proteins by preventing protein-protein interactions that may be involved in the regulation of these proteins or the PI3K/PKB pathway and necessary for enzyme activity. Antibodies with inhibitory activity such as described herein can be produced and identified according to standard assays familiar to one of skill in the art. Therefore, another aspect of the invention relates to the method to prevent, treat, or ameliorate osteoarthritis comprising administering to a subject in need thereof an effective amount of a compound capable of modulating PI3K or PKB and through said modulation alter expression of AGG-1 or COL-3 wherein said PI3K modulator or PKB modulator comprises one or more antibodies to PI3K or PKB, or fragments thereof, wherein said antibodies or fragments thereof can inhibit the enzyme activity of a protein selected from the following: PI3K and PKB.

The invention also provides for methods which employ pharmaceutical compositions to prevent or treat osteoarthritis in a subject by modulating PI3K, PKB, IL-1, or OSM and thus modifying the expressed levels of AGG-1 or COL-3 in said subject. Therefore an aspect of the invention relates to a method to prevent, treat, or ameliorate osteoarthritis comprising administering to a subject in need thereof a pharmaceutical composition comprising an effective amount of a compound capable of modulating PI3K, PKB, IL-1, or OSM and through said modulation alter expression of AGG-1 or COL-3.

Another aspect of the invention relates to the method to prevent, treat, or ameliorate osteoarthritis comprising administering to a subject in need thereof a pharmaceutical composition comprising an effective amount of a compound capable of modulating PI3K, PKB, IL-1, or OSM and through said modulation alter expression of AGG-1 or COL-3 wherein said PI3K modulator, PKB modulator, IL-1 modulator, or OSM modulator inhibits in said subject the enzyme activity of one or more proteins selected from the following: PI3K and PKB.

Yet another aspect of the invention relates to the method to prevent, treat, or ameliorate osteoarthritis comprising administering to a subject in need thereof a pharmaceutical composition comprising an effective amount of a compound capable of modulating PI3K, PKB, IL-1, or OSM and through said modulation alter expression of AGG-1 or COL-3 wherein said PI3K modulator, PKB modulator, IL_{—}1 modulator, or OSM modulator inhibits in said subject gene or protein expression of one or more proteins selected from the following: PI3K, PKB, IL-1, OSM, AGG-1, and COL-3.

In another embodiment the invention relates to the method to prevent, treat, or ameliorate osteoarthritis comprising administering to a subject in need thereof a pharmaceutical composition comprising an effective amount of a compound capable of modulating PI3K or PKB and through said modulation alter expression of AGG-1 or COL-3 wherein said PI3K modulator or PKB modulator is LY249002 2-(4-morpholinyl)-8-phenyl-4H-1-benzopyran-4-one) in free or pharmaceutically acceptable salt forms.

An additional embodiment of the invention relates to the method to prevent, treat, or ameliorate osteoarthritis comprising administering to a subject in need thereof a pharmaceutical composition comprising an effective amount of a compound capable of modulating PI3K, PKB, IL-1, or OSM and through said modulation alter expression of AGG-1 or COL-3 wherein said PI3K modulator, PKB modulator, IL-1 modulator, or OSM modulator comprises one or more antibodies to PI3K or PKB, or fragments thereof wherein said antibodies or fragments thereof can inhibit the enzyme activity of a protein selected from the following: PI3K and PKB.

The invention also provides for a method to screen or identify modulators which inhibit PI3K, PKB, IL-1, or OSM and through that inhibition alter expression of AGG-1 or COL-3. Said modulators are thus useful to prevent, treat, or ameliorate osteoarthritis. Therefore, another aspect of the invention relates to a method to identify modulators useful to prevent, treat, or ameliorate osteoarthritis comprising assaying for the ability of a candidate modulator to inhibit PI3K, PKB, IL-1, or OSM and through said inhibition alter expression of AGG-1 or COL-3.

Conventional screening assays (both in vitro and in vivo) may be used to identify compounds that inhibit PI3K, PKB, IL-1, and OSM protein activity and/or mRNA expression. Protein activity levels can be assayed in a subject using a biological sample e.g., chondrocyte cell lysate, from the subject using conventional enzyme activity assay methods. Gene expression may also be determined using methods familiar to one of skill in the art, including, for example, conventional Northern analysis or commercially available glass chip microarrays. Protein levels may be determined from a biological sample, by methods herein described or by any known method, including immunoassays and electrophoresis assays.

Candidate compounds for analysis according to the methods disclosed herein include chemical compounds known to possess PI3K, PKB, IL-1, OSM and/or inhibitory activity as well as compounds whose effects on these proteins at any level have yet to be characterized. It is contemplated herein that any compound with PI3K, PKB, IL-1, and/or OSM inhibitory activity, not necessarily only those with specific inhibitory activity, may prove to be useful therapeutics.

It will be appreciated by those skilled in the art that a screening assay to locate compounds with effects on PI3K, PKB, IL-1 and/or OSM may comprise techniques familiar to one of skill in the art, for example, an in vitro enzyme activity assay may be employed using conventional methods. Additionally, the effect of test compounds' inhibition of PI3K, PKB, IL-1, and/or OSM and resultant alteration of AGG-1 and/or COL-3 levels can be detected with an ELISA antibody-based assay or fluorescent labelling reaction assay for detection of AGG-1 and/or COL-3. These techniques are readily available for high throughput screening and are familiar to one skilled in the art

The modulators that are discovered in the screening or identification assay may be further validated for their effects in animal models of osteoarthritis in vitro or in vivo and also further assaying for the ability of an identified modulator to reverse the pathological effects observed in animal models of OA and/or in clinical studies with subjects with OA. Therefore, one embodiment of the invention relates to the method to identify modulators useful to prevent, treat, or ameliorate osteoarthritis comprising assaying for the ability of a candidate modulator to inhibit PI3K, PKB, IL-1, or OSM and through said inhibition alter expression of AGG-1 or COL-3 wherein said method further comprises assaying for the ability of an identified PI3K, PKB, IL-1 or OSM inhibitory modulator to reverse the pathological effects observed in animal models of osteoarthritis.

Another aspect of the invention relates to the method to identify modulators useful to prevent, treat, or ameliorate osteoarthritis comprising assaying for the ability of a candidate modulator to inhibit PI3K, PKB, IL-1 or OSM and through said inhibition alter expression of AGG-1 or COL-3 wherein said modulator inhibits the enzyme activity of one or more proteins selected from the following: PI3K and PKB.

Yet another aspect of the invention relates to the method to identify modulators useful to prevent, treat, or ameliorate osteoarthritis comprising assaying for the ability of a candidate modulator to inhibit PI3K, PKB, IL-1, or OSM and through said inhibition alter expression of AGG-1 or COL-3 wherein said modulator inhibits gene or protein expression of one or more proteins selected from the following: PI3K, PKB, IL-1, OSM, AGG-1, and COL-3

The invention also provides that modulators which are discovered in the screening or identification assay above may be further validated for their effects in clinical studies of subjects who have active or nascent osteoarthritis. Therefore, an additional aspect of the invention relates to the method to identify modulators useful to prevent, treat, or ameliorate osteoarthritis comprising assaying for the ability of a candidate modulator to inhibit PI3K, PKB, IL-1, or OSM and through said inhibition alter expression of AGG-1 or COL-3 wherein said method further comprises assaying for the ability of an identified inhibitory modulator to reverse the pathological effects observed in clinical studies with subjects with osteoarthritis.

Another aspect of the invention relates to the method to identify modulators useful to prevent, treat, or ameliorate osteoarthrtis comprising assaying for the ability of a candidate modulator to inhibit PI3K, PKB, IL-1 or OSM and through said inhibition alter expression of AGG-1 or COL-3 wherein said PI3K modulator, PKB modulator, IL-1 modulator, or OSM modulator comprises any one or more substances selected from the following: antisense oligonucleotides, triple helix DNA, single stranded DNA, ribozymes, RNA aptamer and double stranded RNA wherein said substances are designed to inhibit gene expression of PI3K, PKB, IL-1, OSM, AGG-1 or COL-3 proteins.

In another embodiment the invention relates to the method to identify modulators useful to prevent, treat, or ameliorate osteoarthritis comprising assaying for the ability of a candidate modulator to inhibit PI3K, PKB, IL-1, OSM and through said inhibition alter expression of AGG-1 or COL-3 wherein said PI3K modulator, PKB modulator, IL-1 modulator, or OSM modulator comprises one or more antibodies to PI3K, PKB, IL-1, or OSM or fragments thereof, wherein said antibodies or fragments thereof can inhibit the enzyme activity of a protein selected from the following: PI3K and PKB.

The invention provides for pharmaceutical compositions comprising a PI3K modulator, PKB modulator, IL-1 modulator, or OSM modulator in an amount effective to prevent, treat, or ameliorate osteoarhtritis in a subject in need thereof.

Another aspect of the invention relates to the pharmaceutical composition comprising a PI3K modulator, PKB modulator, IL-1 modulator, or OSM modulator in an amount effective to prevent, treat, or ameliorate osteoarhtritis in a subject in need thereof wherein said modulator inhibits the enzyme activity of one or more proteins selected from the following: PI3K and PKB.

Yet another aspect of the invention relates to the pharmaceutical composition comprising a PI3K modulator, PKB modulator, IL-1 modulator, or OSM modulator in an amount effective to prevent, treat, or ameliorate osteoarhtritis in a subject in need thereof wherein said modulator inhibits gene or protein expression of one or more proteins selected from the following: PI3K, PKB, IL-1, OSM, AGG-1, and COL-3.

One embodiment of the invention relates to the pharmaceutical composition comprising a PI3K modulator or PKB modulator in an amount effective to prevent, treat, or ameliorate osteoarhtritis in a subject in need thereof wherein said modulator is LY249002 2-(4-morpholinyl)-8-phenyl-4H-1-benzopyran-4-one) in free or pharmaceutically acceptable salt forms.

Another embodiment of the invention relates to the pharmaceutical comprising a PI3K modulator, PKB modulator, IL-1 modulator, or OSM modulator in an amount effective to prevent, treat, or ameliorate osteoarhtritis in a subject in need thereof wherein said modulator comprises any one or more substances selected from the following: antisense oligonucleotides, triple helix DNA, single stranded DNA, ribozymes, RNA aptamer and double stranded RNA wherein said substances are designed to inhibit gene expression of PI3K, PKB, IL-1, OSM, AGG-1 or COL-3 proteins.

Yet another embodiment of the invention relates to the pharmaceutical composition comprising a PI3K modulator, PKB modulator, IL-1 modulator, or OSM modulator in an amount effective to prevent, treat, or ameliorate osteoarhtritis in a subject in need thereof wherein said modulator comprises one or more antibodies to PI3K, PKB, IL-1, or OSM, or fragments thereof, wherein said antibodies or fragments thereof can inhibit the enzyme activity of a protein selected from the following: PI3K and PKB.

Polynucleotides, nucleotides, polypeptides, and antibodies of the present invention may also be used diagnostically. For example, one could use said antibodies according to conventional methods to quantitate levels of PI3K, PKB, IL-1, OSM, AGG-1 or COL-3 in a subject; increased levels would indicate the degree of severity of OA. Thus, different protein levels would be indicative of various clinical forms or severity of OA. Such information would also be useful to identify subsets of patients experiencing arthritis that may or may not respond to treatment with the modulators of the present invention. Similarly, it is contemplated herein that quantitating the message level of PI3K, PKB, IL-1, OSM, AGG-1 or COL-3 in a subject would be useful for diagnosis and determining appropriate OA therapy; subjects with increased mRNA levels of PI3K, PKB, IL-1, OSM, AGG-1 or COL-3 compared to appropriate control individuals would be considered suitable candidates for treatment with PI3K, PKB, IL-1, and/or OSM inhibitors.

The invention provides for the ability of polynucleotides, nucleotides, polypeptides, and antibodies of the present invention to be used diagnostically. Therefore, in one embodiment the invention relates to a method to diagnose subjects affected with active or nascent osteoarthritis who may be suitable candidates for treatment with PI3K, PKB, IL-1, or OSM modulators comprising assaying mRNA levels of a substance selected from one or more of the following: α,β,γ, and δ isoforms of PI3K, IL-1, OSM, AGG-1, and COL-3 in a biological sample from said subject wherein subjects with altered, such as either increased or decreased, levels of said PI3K, IL-1, OSM, AGG-1, or COL-3 compared to non-osteoarthritic control levels would be suitable candidates for PI3K modulator, PKB modulator, IL-1 modulator, or OSM modulator treatment. Said method of diagnosis may be intended to identify those subjects with active or nascent osteoarthritis as those subjetcs who have altered levels of said PI3K, IL-1, OSM, AGG-1, or COL-3 compared to the levels observed in non-osteoarthritic control biological samples.

Another aspect of the invention contemplates and provides for detection of the ratios of α,β,γ, and δ isoforms of PI3K in a subject for diagnosis of nascent or active OA. For example, antibodies to the PI3K isoforms α,β,γ, and δ could be used to detect the ratios of α,β,γ, and δ isoforms of PI3K in a subject. The ratio of these isoforms to one another is characteristically altered in osteoarthritic chondrocytes compared to the ratios of these proteins in normal chondrocytes as shown in Tables 6 through 10. Comparisons of the ratios of these proteins in articular chondrocyte to those of levels seen in patients who have active or nascent OA can be used to monitor PI3K and PI3K isoforms, PKB, IL-1, OSM, AGG-1 and/or COL-3 levels as part of a clinical testing procedure, e.g., for example, to determine the efficacy of a given treatment regimen or to aid in diagnosis of ostearthritic disorders. Interestingly, chondrocytes may be obtained from an osteoarthritic joint as herein described and cultured in the presence of PDGF, which is an inducer of PI3K expression. The detection of α,β,γ, and δ isoforms of PI3K can then be performed for example, using Western blotting detection methods or via fluorescent labelled antibodies to the isoforms.

In another aspect, the invention provides for an assay capable of assessing the ability of an identified inhibitory substance to reverse the pathological effects observed in animal models of OA and/or in clinical studies with subjects with OA and are well known to a practitioner of ordinary skill in the art. For example, one animal model in which inhibitors of PI3K, PKB, IL-1, and/or OSM can be tested for therapeutic efficacy in OA is the spontaneous OA model in which the animals to be tested are known to spontaneously generate osteoarthrtitic lesions. An example of the spontaneous animal model of osteoarthritis is found in the STR/ort mouse model. Mason, R. M., et al., “The STR/ort mouse and its Use as a Model of Osteoarthritis” Osteoarthritis and Cartilage (2001) 9: 85-91. An additional example of the spontaneous generation of osteoarthritis is found in Dunkin Hartley guinea pigs. The use of Dunkin Hartley guinea pigs for a spontaneous animal model of oseoarthritis is described in Jimenez, P. A., et al., “Spontaneous Osteoarthritis in Dunkin Hartley guinea pigs: Histologic, Radiologic and Biochemical Changes” Lab. Anim. Sci. (1997) 27: 134-142.

Double blinded placebo or active comparator controlled clinical studies may also be designed to test the effects in osteoarthritis patients of inhibitors discovered with the inhibitor screen of the invention.

### DETAILED DESCRIPTION OF THE INVENTION

Abbreviations

PI3K, phosphoinositide 3-kinase;
PKB, protein kinase B;
IL-1, Interleukin-1;
TNF, tumor necrosis factor;
OA, osteoarthritis;
RA, rheumatoid arthritis;
AGG-1, aggrecanase-1;
COL-3, collagenase-3;
PDGF, platelet-derived growth factor;
MMP, matrix metalloproteinase;
OSM, oncostatin M;
GAPDH, glyceraldehyde 3-phosphate dehydrogenase;
ADAMTS, A Disintegrin And Metalloproteinase with Thrombospondin motifs;
FKHR, forkhead rhabdomyosarcoma;
PCR, polymerase chain reaction;
RT-PCR, reverse transcriptase polymerase chain reaction;
   Definitions

It is contemplated that the invention described herein is not limited to the particular methodology, protocols, and reagents described as these may vary. It is also to be understood that the terminology used herein is for the purpose of describing particular embodiments only, and is not to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology, microbiology, and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled In the art, and so forth.

A “vector” molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.”

The ability of a substance to “modulate” PI3K, PKB, IL-1, OSM, AGG-1 or COL-3 includes, but is not limited to, the ability of a substance to inhibit the enzymatic activity of these proteins and/or inhibit gene expression of these proteins. Such modulation could also involve effecting the ability of other proteins to interact with these proteins, for example related regulatory proteins or proteins that are modified by PI3K, PKB, IL-1, OSM, AGG-1 or COL-3. A “modulator” of PI3K, PKB, IL-1, OSM, AGG-1 or COL-3 refers to a substance that can have these effects, among others, on these proteins or, e.g., related regulatory proteins.

Similarly, the terms “antagonist” or “inhibitor” as used herein, refer to a molecule which when bound to PI3K, PKB, IL-1, or OSM blocks or modulates the biological activity these proteins with a resultant down-regulation of AGG-1 and COL-3 in cartilage or chondrocytes. Antagonists and inhibitors may include proteins, nucleic acids, carbohydrates, or any other molecules, natural or synthetic that bind to these polypeptides.

“Nucleic acid sequence”, as used herein, refers to an oligonucleotide, nucleotide or polynucleotide, and fragments or portions thereof, and to DNA or RNA of genomic or synthetic origin that may be single or double stranded, and represent the sense or antisense strand.

The term “antisense” as used herein, refers to nucleotide sequences which are complementary to a specific DNA or RNA sequence. The term “antisense strand” is used in reference to a nucleic acid strand that is complementary to the “sense’ strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative” is sometimes used in reference to the antisense strand, and “positive” is sometimes used in reference to the sense strand.

As contemplated herein, antisense oligonucleotides, triple helix DNA, RNA aptamers, ribozymes and double stranded RNA are directed to a nucleic acid sequence of PI3K, PKB, IL-1, OSM, AGG-1 or COL-3 such that the nucleotide sequence of these proteins chosen will produce gene-specific inhibition of gene expression of these proteins. For example, knowledge of the PI3K nucleotide sequence may be used to design an antisense molecule which gives strongest hybridization to the mRNA. Similarly, ribozymes can be synthesized to recognize specific nucleotide sequences of PI3K, PKB, IL-1, OSM, AGG-1 or COL-3 and cleave them (Cech. J. Amer. Med Assn. 260:3030 (1988). Techniques for the design of such molecules for use in targeted inhibition of gene expression is well known to one of skill in the art.

The terms “PI3K, PKB, IL-1, OSM, AGG-1 or COL-3” refer to any and all forms of these polypeptides including, but not limited to, variants, partial forms, isoforms, precursor forms, the full length polypeptide, fusion proteins containing PI3K, PKB, IL-1, OSM, AGG-1 or COL-3 sequences or fragments of any of the above, from human or any other species. Isoforms of PI3K include PI3 Kα, PI3 Kβ, PI3K,γ, and PI3Kδ. The sequence of human PI3Kα may be found in Genbank, Accession Number Z29090. The sequence of human PI3Kβ may be found in Genbank, Accession Number S67334. The sequence of human PI3Kγ may be found in Genbank, Accession Number X83368. The sequence of human PI3KS may be found in Genbank, Accession Number U86453. The sequence of murine PKB may be found in Genbank, Accession Number NM-009652. The sequence of human interleukin-1 beta may be found in the NCBI Genbank, Accession Number NP 000567. The sequence of human oncostatin M may be found in the NCBI Genbank, Accession Number NP 065391. The sequence of human AGG-1 may be found in Genbank, Accession Number Af148213. The sequence of human COL-3 may be found in Genbank, Accession Number XM_{—}006274. Homologs, as well as human orthologs, of these proteins, which would be apparent to one of skill in the art, are meant to be included in this definition. It is also contemplated that the term refers to these proteins isolated from naturally occurring sources of any species such as genomic DNA libraries as well as genetically engineered host cells comprising expression systems, or produced by chemical synthesis using, for instance, automated peptide synthesizers or a combination of such methods. Means for isolating and preparing such polypeptides are well understood in the art.

The term “sample” as used herein, is used in its broadest sense. A biological sample from a subject may comprise cartilage, blood, urine or other biological material with which PI3K, PKB, IL-1, OSM, AGG-1 or COL-3 enzymatic activity or gene expression may be assayed. A biological sample may include articular cartilage from which total RNA may be purified for gene expression profiling using conventional glass chip microarray technologies such as Affymetrix chips, RT-PCR or other conventional methods.

As used herein, the term “antibody” refers to intact molecules as well as fragments thereof, such as Fa, F(ab′)₂, and Fv, which are capable of binding the epitopic determinant. Antibodies that bind PI3K, PKB, IL-1, OSM, AGG-1 or COL-3 polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat or a rabbit).

The term “humanized antibody” as used herein, refers to antibody molecules in which amino acids have been replaced in the non-antigen binding regions in order to more closely resemble a human antibody, while still retaining the original binding ability.

A “therapeutically effective amount” is the amount of a drug sufficient to treat and/or ameliorate the pathological effects of OA, including but not limited to, joint pain, decreased mobility and range of joint motion, immobile or frozen joints, progressive degradation of articular cartilage, and decreased joint strength.

“Related regulatory proteins” and “related regulatory polypeptides” as used herein refer to polypeptides involved in the regulation of PI3K, PKB, IL-1, OSM, AGG-1 or COL-3 which may be identified by one of skill in the art using conventional methods such as described herein.

OA and “related conditions” includes but is not limited to those disease states and conditions which are either medically definable or diagnosable as osteoarthritis or are medically identifiable as accompanying complications or conditions which are either derivative of ostearthritic disease or are associated sequelae. “Nascent OA” refers to early stages of osteoarthritis which may be diagnosable due to observable mild symptoms of osteoarthritis such as, for example, joint pain and stiffness. Nascent OA may also be characterized as asymptomatic but diagnosable through means such as, for example, an x-ray indicating evidence of degenerative joint space narrowing, biochemical assay of articular chondrocytes for altered and/or increased levels of PI3K and/or isoforms thereof, and biochemical assay of articular chondrocytes for altered and/or increased levels of AGG-1 and/or COL-3. “Active OA” refers to arthritic disease which is symptomatic and either medically diagnosable or diagnosed positively as osteoarthritis by a physician. An osteoarthritic disorder refers to those conditions or states which are cause by nascent or active osteoarthritis.

“Subject” and or “patient” refers to any human or nonhuman organism.

As used herein, a “transformation vector” is one that uses the transposable element technique to mediate integration of a piece of DNA in the genome of the organism and is familiar to one of skill in the art.

The terms “candidate compound” and “compound” as used herein, refer to a molecule which when bound to either PI3K, PKB, IL-1, or OSM blocks or modulates the biological activity of PI3K or PKB with a resultant down-regulation of AGG-1 and/or COL-3. Candidate compounds and compounds may include proteins, nucleic acids, carbohydrates, or any other molecules, natural or synthetic that bind to said polypeptide.

Description

The levels of the matrix degrading enzymes AGG-1 and COL-3 are increased in osteoarthritic cartilage versus normal cartilage. These altered levels of AGG-1 And COL-3 proteases may contribute to the initiation and progression of osteoarthritis.

The current invention relates to the discovery that IL-1 and OSM are suitable targets for the development of new therapeutics to treat, prevent or ameliorate OA through a new and distinct mechanism of action. In particular, the applicants have surprisingly discovered that altering, inhibiting, or modulating the enzyme activity or gene expression of IL-1 and OSM in vitro or in vivo, leads to a downregulation of PI3K and/or PKB activated AGG-1 and/or COL-3 gene and/or protein expression and/or secretion. Increased levels of the matrix degrading enzymes AGG-1 and COL-3 are implicated in OA. Therefore, inhibitors of IL-1 and OSM would operate as compounds useful for treatment of OA through the newly elucidated intermediary mechanism of IL-1 and OSM mediated PI3K and/or PKB activation of AGG-1 and COL-3 production.

The invention is based on the discovery that PI3K, PKB, IL-1, and OSM activation leads to AGG-1 and COL-3 expression and/or production. Given the destructive role of AGG-1 and COL-3 in the pathogenesis of OA, PI3K and PKB and isoforms thereof may be used as novel drug targets for OA. Thus, it is contemplated herein that PI3K and PKB are useful drug targets for the development of therapeutics to treat, prevent or ameliorate OA, a disease state not previously known to involve the activation of AGG-1 and COL-3 by PI3K, PKB, IL-1, and OSM activation.

In one embodiment, the present invention provides a method to prevent, treat, or ameliorate a disease associated with or caused by altered levels of AGG-1 or COL-3. This embodiment comprises administering to a subject or patient who may have any disease associated with increased levels of AGG-1 and/or COL-3, an effective amount of a compound capable of modulating PI3K, PKB, IL-1, or OSM, genes and/or proteins, including, but not limited to, increasing or decreasing the articular levels of these substances, and through said modulation alter gene and/or protein expression and/or secretion of AGG-1 or COL-3. Said administered PI3K, PKB, IL-1, and/or OSM modulating compound is intended to reduce the levels of AGG-1 and/or COL-3 genes and/or proteins and lead to an improvement in the associated disease state in the subject or patient. The intended disease improvement may be evidenced by methods such as, including but not limited to, biochemical detection through an assay to determine AGG-1 and/or COL-3 gene and/or protein levels in affected bodily tissues or subjective improvement in disease symptoms.

Increased levels of AGG-1 and or COL-3 have been correlated with OA. Therefore, another aspect of the invention relates to a method to prevent, treat, or ameliorate osteoarthritis, by administering to a subject in need thereof, an effective amount of a compound capable of modulating PI3K, PKB, IL-1, and/or OSM genes and/or proteins, including, but not limited to, increasing or decreasing the articular levels of these substances, and through said modulation alter gene and/or protein expression and/or secretion of AGG-1 or COL-3. This embodiment comprises administering to a subject or patient, who may have active or nascent OA, an effective amount of a compound capable of modulating PI3K, PKB, IL-1, or OSM and through said modulation alter the levels of AGG-1 or COL-3. Said administered PI3K, PKB, IL-1, and/or OSM modulating compound is intended to reduce the levels of AGG-1 and/or COL-3 genes and/or proteins and lead to an improvement in OA in the subject or patient. The OA improvement may be evidenced by methods such as, including but not limited to, biochemical detection through an assay to determine AGG-1 and/or COL-3 levels in articular chondrocytes or subjective improvement in OA symptoms.

It is also contemplated that prevention, treatment, or amelioration of OA may also be accomplished by reducing AGG-1 and/or COL-3 levels through administration of a PI3K, PKB, IL-1, and/or OSM modulator that is specifically intended to reduce or inhibit the enzyme activity of PI3K and/or PKB. Another aspect of the invention, therefore, relates to a method to prevent, treat or ameliorate osteoarthritis wherein a subject who has nascent or active OA is administered an effective amount of a compound capable of inhibiting PI3K or PKB enzyme activity. The inhibition of PI3K and/or PKB enzyme activity is further intended to reduce gene expression or protein levels of AGG-1 or COL-3 and lead to improvement of OA in the afflicted individual. Improvement of nascent or active OA in the subjects receiving such treatment may be monitored by an assay to determine AGG-1 and/or COL-3 levels in articular chondrocytes or subjective improvement in OA symptoms.

In another interesting embodiment, the invention relates to a method to prevent, treat, or ameliorate osteoarthritis by inhibiting gene and/or protein expression of PI3K, PKB, IL-1, OSM, AGG-1, and/or COL-3. This method includes administering to a subject, with nascent or active osteoarthritis, an effective amount of a compound that is a PI3K modulator or PKB modulator with the purpose of inhibiting any of the genes or proteins PI3K, PKB, IL-1, OSM, AGG-1, and COL-3. Therefore, it is contemplated that the effect of the PI3K modulator, PKB modulator, IL-1 modulator, and/or OSM modulator in this embodiment may inhibit gene and/or protein expression of any or all of the four proteins, PI3K, PKB, IL-1, OSM, AGG-1, and COL-3, that have been correlated with the disease pathway identifed in the current invention.

The discovery that a PI3K and/or PKB inhibitor, alters expression of AGG-1 or COL-3 is novel and constitutes a key aspect of the invention. A known PI3K and/or PKB inhibitor, includes a compound referred to herein as LY290042 (also known as 2-(4-morpholinyl)-8-phenyl-4H-1-benzopyran-4-one), (Vlahos, C J, et al. J. Biol. Chem. (1994) 269: 7 5241-5248) and commercially available from Sigma-Aldrich, St. Louis, Mo. Based on the surprisingly good results, observed as the reduction of levels of AGG-1 and COL-3, it is contemplated herein that a pharmaceutical composition comprising this compound in free or pharmaceutically acceptable salts may be used to inhibit PI3K and/or PKB and through this inhibition reduce AGG-1 and COL-3 mRNA, gene, and/or protein levels in cartilage and/or chondrocytes. Therefore LY294002 base or its salts are useful to treat, prevent or ameliorate OA in a subject via the inhibition of PI3K and/or PKB and corresponding reduction in AGG-1 and/or COL-3 levels in affected bodily tissues.

Modulation of gene expression of PI3K, PKB, IL1, OSM, AGG-1, and/or COL-3 will correspondingly affect the levels of these substances in a subject. Therefore, it is contemplated that certain biological tools that are directed to modulate gene expression of PI3K, PKB, IL-1, and/or OSM could also inhibit gene expression of PI3K, PKB, IL-1, OSM, AGG-1, and/or COL-3. Such biological tools that inhibit gene expression would be useful for treatment of OA. Therefore, another interesting aspect of the invention utilizes a PI3K modulator, PKB modulator, IL-1 modulator, OSM modulator comprising any one or more of antisense oligonucleotides, triple helix DNA, single stranded DNA, ribozymes, RNA aptamer and double stranded RNA wherein these substances are designed to inhibit gene expression of PI3K, PKB, IL-1, OSM, AGG-1 or COL-3 proteins and benefit a subject with nascent or active OA.

Suitable antibodies to PI3K, PKB, IL-1, and or OSM or related regulatory proteins are capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above

For the production of antibodies to the polypeptides discussed herein, various host animals may be immunized by injection with the polypeptides, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the polypeptides, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Detection of the antibodies described herein may be achieved using standard ELISA, FACS analysis, and standard imaging techniques used in vitro or in vivo. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I ³⁵S or ³H.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the PI3K, PKB, IL-1, or OSM polypeptide or related regulatory protein, or fragments thereof.

The most commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of polypeptide or polypeptide fragment of interest which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalzye endonucleolytic cleavage of sequences encoding the genes of the polypeptides for PI3K, PKB, IL-1, OSM, AGG-1, COL-3 or various regulatory proteins involved in its upregulation.

Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stern and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Gene specific inhibition of gene expression may also be achieved using conventional double stranded RNA technologies. A description of such technology may be found in WO 99/32619 which is hereby incorporated by reference in its entirety.

Antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well known in the art.

It is contemplated herein that one can inhibit the function and/or expression of a gene for a related regulatory protein or protein modified by PI3K, PKB, IL-1, OSM, AGG-1, COL-3 as a way to treat OA by designing, for example, antibodies to these proteins and/or designing inhibitory antisense oligonucleotides, triple helix DNA, ribozymes and RNA aptamers targeted to the genes for such proteins according to conventional methods. Pharmaceutical compositions comprising such inhibitory substances for the treatment of OA are also contemplated.

An interesting embodiment of the invention relates to pharmaceutical compositions which comprise antibodies that are highly selective for human PI3K, PKB, IL-1, OSM, AGG-1 and/or COL-3 polypeptides or portions of human PI3K, PKB, IL-1, OSM, AGG-1 and/or COL-3 polypeptides. Antibodies to these proteins may cause the aggregation of these proteins in a subject and thus reduce the activity of the PI3k, PKB, AGG-1, and/or COL-3 enzymes. Such antibodies may also decrease enzymatic activity, for example, by interacting directly with active sites or by blocking access of substrates to active sites. PI3K, PKB, IL-1, and/or OSM antibodies may also be used to inhibit enzymatic activity of these proteins by preventing protein-protein interactions that may be involved in the regulation of these proteins or the PI3K/PKB pathway and necessary for enzyme activity. Antibodies with inhibitory activity such as described herein can be produced and identified according to standard assays familiar to one of skill in the art. Therefore, another aspect of the invention relates to a method to prevent, treat, or ameliorate osteoarthritis by administering, to a subject with OA, an effective amount of an antibody or active antibody fragment to PI3K, PKB, IL-1, and/or OSM. The antibody thus used is intended to inhibit the enzyme activity of PI3K and PKB and correspondingly reduce the levels of AGG-1 and/or COL-3.

The pharmaceutical compositions of the present invention may comprise substances that inhibit the expression of PI3K, PKB, IL-1, and/or OSM at the nucleic acid level. Such molecules include ribozymes, antisense oligonucleotides, triple helix DNA, RNA aptamers and/or double stranded RNA directed to an appropriate nucleotide sequence of PI3K, PKB, IL-1, OSM, AGG-1, COL-3 or nucleic acid or a related regulatory polypeptide of interest. These may be created using conventional techniques by one of skill in the art without undue burden or experimentation. For example, modifications (e.g. inhibition) of gene expression can be obtained by designing antisense molecules, DNA or RNA, to the control regions of the genes encoding the polypeptides discussed herein, i.e. the promoters, enhancers, and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

The invention also provides for a method to screen or identify modulators which inhibit PI3K, PKB, IL-1, or OSM and through that inhibition alter expression of AGG-1 or COL-3. Said modulators are thus useful to prevent, treat, or ameliorate osteoarthritis. Therefore, another aspect of the invention relates to a method to identify modulators useful to prevent, treat, or ameliorate osteoarthritis comprising assaying for the ability of a candidate modulator to inhibit PI3K, PKB, IL-1, or OSM and through said inhibition alter expression of AGG-1 or COL-3.

Conventional screening assays (both in vitro and in vivo) may be used to identify compounds that inhibit PI3K, PKB, IL-1, and OSM protein activity and/or mRNA expression. Protein activity levels can be assayed in a subject using a biological sample e.g., chondrocyte cell lysate, from the subject using conventional enzyme activity assay methods. Gene expression may also be determined using methods familiar to one of skill in the art, including, for example, conventional Northern analysis or commercially available glass chip microarrays. Protein levels may be determined from a biological sample, by methods herein described or by any known method, including immunoassays and electrophoresis assays.

Candidate compounds for analysis according to the methods disclosed herein include chemical compounds known to possess PI3K, PKB, IL-1, and/or OSM inhibitory activity as well as compounds whose effects on these proteins at any level have yet to be characterized. It is contemplated herein that any compound with PI3K, PKB, IL-1 and/or OSM inhibitory activity, not necessarily only those with specific inhibitory activity, may prove to be useful therapeutics.

It will be appreciated by those skilled in the art that a screening assay to locate compounds with effects on PI3K, PKB, IL-1, and/or OSM may comprise techniques familiar to one of skill in the art, for example, an in vitro enzyme activity assay may be employed using conventional methods. Additionally, the effect of test compounds' inhibition of PI3K, PKB, IL-1, and/or OSM and resultant alteration of AGG-1 and/or COL-3 levels can be detected with an ELISA antibody-based assay or fluorescent labelling reaction assay for detection of AGG-1 and/or COL-3. These techniques are readily available for high throughput screening and are familiar to one skilled in the art

Modulators may have a positive effect on the levels of PI3K, PKB, IL-1, and/or OSM even if such modulators correspondingly reduce the levels of AGG-1 and/or COL-3, but do not neccesarily inhibit PI3K, PKB, IL-1, and/or OSM. Such modulators may alter the activation states of the PI3K isoforms and pathways so that the modulated PI3K and/or PKB enzyme activity or modulated IL-1 or OSM activity does not lead to a stimulation of AGG-1 and/or COL-3. Therefore, another aspect of the invention relates to a method to identify PI3K and/or PKB modulators that are useful to prevent, treat, or ameliorate osteoarthritis comprising assaying for the ability of a candidate modulator to inhibit PI3K, PKB, IL-1, or OSM and through said inhibition alter expression of AGG-1 or COL-3.

The modulators that are discovered in a screening or identification assay may be further validated for their effects in animal or human clinical models of osteoarthritis in vitro or in vivo and also further assaying for the ability of an identified modulator to reverse the pathological effects observed in animal or models of OA and/or in clinical studies with subjects with OA. Therefore, one embodiment of the invention relates to a method to further test PI3K, PKB, IL-1, and/or OSM modulators that are identified in a screening or identification assay useful to prevent, treat, or ameliorate osteoarthritis comprising assaying for the ability of identified PI3K modulators, PKB modulators, IL-1 modulators, and/or OSM modulators which correspondingly affect AGG-1 and/or COL-3 gene and/or protein expression, for their ability to reverse the pathological effects observed in animal models or clinical studies of subjects with OA.

Yet another aspect of the invention relates to the method to identify modulators useful to prevent, treat, or ameliorate osteoarthritis comprising assaying for the ability of a candidate modulator to inhibit PI3K, PKB, IL-1, or OSM and through said inhibition alter expression of AGG-1 or COL-3 wherein said modulator inhibits gene or protein expression of one or more proteins selected from the following: PI3K, PKB, IL-1, OSM, AGG-1, and COL-3

The invention also provides that modulators which are discovered in the screening or identification assay above may be further validated for their effects in clinical studies of subject s who have active or nascent osteoarthritis. Therefore, an additional aspect of the invention relates to the method to identify modulators useful to prevent, treat, or ameliorate osteoarthritis comprising assaying for the ability of a candidate modulator to inhibit PI3K, PKB, IL-1, or OSM and through said inhibition alter expression of AGG-1 or COL-3 wherein said method further comprises assaying for the ability of an identified inhibitory modulator to reverse the pathological effects observed in clinical studies with subjects with osteoarthritis.

Another aspect of the invention relates to the method to identify modulators useful to prevent, treat, or ameliorate osteoarthritis comprising assaying for the ability of a candidate modulator to inhibit PI3K, PKB, IL-1 or OSM and through said inhibition alter expression of AGG-1 or COL-3 wherein said PI3K modulator, PKB modulator, IL-1 modulator, or OSM modulator comprises any one or more substances selected from the following: antisense oligonucleotides, triple helix DNA, single stranded DNA, ribozymes, RNA aptamer and double stranded RNA wherein said substances are designed to inhibit gene expression of PI3K, PKB, IL-1, OSM, AGG-1 or COL-3 proteins.

In another embodiment the invention relates to the method to identify modulators useful to prevent, treat, or ameliorate osteoarthritis comprising assaying for the ability of a candidate modulator to inhibit PI3K, PKB, IL-1, or OSM and through said inhibition alter expression of AGG-1 or COL-3 wherein said PI3K modulator, PKB modulator, IL-1 modulator, or OSM modulator comprises one or more antibodies to PI3K, PKB, IL-1, or OSM, or fragments thereof, wherein said antibodies or fragments thereof can inhibit the enzyme activity PI3K or PKB.

The invention provides for pharmaceutical compositions comprising a PI3K modulator, PKB modulator, IL-1 modulator, or OSM modulator in an amount effective to prevent, treat, or ameliorate osteoarhtritis in a subject in need thereof.

Another aspect of the invention relates to the pharmaceutical composition comprising a PI3K modulator, PKB modulator, IL-1 modulator, or OSM modulator in an amount effective to prevent, treat, or ameliorate osteoarhtritis in a subject in need thereof wherein said modulator inhibits the enzyme activity of one or more proteins selected from the following: PI3K and PKB.

Yet another aspect of the invention relates to the pharmaceutical composition comprising a PI3K modulator or PKB modulator in an amount effective to prevent, treat, or ameliorate osteoarthritis in a subject in need thereof wherein said modulator inhibits gene or protein expression of one or more proteins selected from the following: PI3K, PKB, IL-1 OSM, AGG-1, and COL-3.

One embodiment of the invention relates to the pharmaceutical composition comprising a PI3K modulator or PKB modulator in an amount effective to prevent, treat, or ameliorate osteoarthritis in a subject in need thereof wherein said modulator is LY249002 2-(4-morpholinyl)-8-phenyl-4H-1-benzopyran-4-one) in free or pharmaceutically acceptable salt forms.

Another embodiment of the invention relates to the pharmaceutical comprising a PI3K modulator, PKB modulator, IL-1 modulator, or OSM modulator, in an amount effective to prevent, treat, or ameliorate osteoarthritis in a subject in need thereof wherein said modulator comprises any one or more substances selected from the following: antisense oligonucleotides, triple helix DNA, single stranded DNA, ribozymes, RNA aptamer and double stranded RNA wherein said substances are designed to inhibit gene expression of PI3K, PKB, IL-1, OSM, AGG-1 or COL-3 proteins.

Yet another embodiment of the invention relates to the pharmaceutical composition comprising a PI3K modulator, PKB modulator, IL-1 modulator, or OSM modulator in an amount effective to prevent, treat, or ameliorate osteoarthritis in a subject in need thereof wherein said modulator comprises one or more antibodies to PI3K, PKB, IL-1, or OSM, or fragments thereof, wherein said antibodies or fragments thereof can inhibit the enzyme activity of a protein selected from the following: PI3K and PKB.

Individuals affected with OA may be difficult to diagnose by traditional conventional methods such as x-rays and subjective symptoms. OA can be asymptomatic and symptomatic in alternating intervals throughout the disease state. Additionally, those subjects with nascent OA may be asymptomatic for the majority of the time until their disease progresses to a greatly deteriorated state. Early detection and treatment or prevention of OA could prevent the extreme physical disability associated with later stages of the disease. A biochemical means to test for nascent or active OA would aid early detection and disease confirmation.

Polynucleotides, nucleotides, polypeptides, and antibodies of the present invention may be used diagnostically. For example, one could use said antibodies according to conventional methods to quantitate levels of PI3K, PKB, IL-1, OSM, AGG-1 or COL-3 in a subject; increased levels would indicate the degree of severity of OA. Thus, different protein levels would be indicative of various clinical forms or severity of OA. Such information would also be useful to identify subsets of patients experiencing arthritis that may or may not respond to treatment with the modulators of the present invention. Similarly, it is contemplated herein that quantitating the message level of PI3K, PKB, IL-1, OSM, AGG-1 or COL-3 in a subject would be useful for diagnosis and determining appropriate OA therapy; subjects with increased mRNA levels of PI3K, PKB, IL-1, OSM, AGG-1 or COL-3 compared to appropriate control individuals would be considered suitable candidates for treatment with PI3K, PKB, IL_{—}1, and/or OSM inhibitors.

The invention provides for the ability of polynucleotides, nucleotides, polypeptides, and antibodies of the present invention to be used diagnostically. Therefore, in one embodiment the invention relates to a method to diagnose subjects affected with active or nascent osteoarthritis who may be suitable candidates for treatment with PI3K, PKB, IL-1, or OSM modulators comprising assaying mRNA levels of a substance selected from one or more of the following: α,β,γ, and δ isoforms of PI3K, IL-1, OSM, AGG-1, and COL-3 in a biological sample from said subject wherein subjects with altered, such as either increased or decreased, levels of PI3K, IL-1, OSM, AGG-1, or COL-3 compared to non-osteoarthritic control levels would be suitable candidates for PI3K modulator or PKB modulator treatment. Said method of diagnosis may be intended to identify those subjects with active or nascent osteoarthritis as those subjects who have altered levels of PI3K, IL-1, OSM, AGG-1, or COL-3 compared to the levels observed in non-osteoarthritic control biological samples.

Another aspect of the invention contemplates and provides for detection of the ratios of α,β,γ, and δ isoforms of PI3K in a subject for diagnosis of nascent or active OA. For example, antibodies to the PI3K isoforms α,β,γ, and δ could be used to detect the ratios of α,β,γ, and δ isoforms of PI3K in a subject. The ratio of these isoforms to one another is characteristically altered in osteoarthritic chondrocytes compared the ratios of these proteins in normal chondrocytes. Comparisons of the ratios of these proteins in articular chondrocyte to those of levels seen in patients who have active or nascent OA can be used to monitor PI3K and PI3K isoforms, PKB, IL-1, OSM, AGG-1 and/or COL-3 levels as part of a clinical testing procedure, e.g., for example, to determine the efficacy of a given treatment regimen or to aid in diagnosis of osteoarthritic disorders. Interestingly, chondrocytes may be obtained from an osteoarthritic joint as herein described and cultured in the presence of PDGF, which is an inducer of PI3K expression. The detection of α,β,γ, and δ isoforms of PI3K can then be performed for example, using Western blotting detection methods or via fluorescent labeled antibodies to the isoforms.

Thus, in addition to the use of these antibodies for the inhibition of enzyme activity or to prevent protein-protein interactions that may be involved in the up regulation of PI3K, PKB, IL-1, OSM, AGG-1 and/or COL-3, such antibodies may be used diagnostically, for example, as a way to monitor PI3K and differential PI3K isoform levels, PKB, IL-1, OSM, AGG-1 and/or COL-3 levels and thus can be used to characterize subjects with osteoarthritis.

Therefore, another aspect of the invention relates to a method to prevent, treat, or ameliorate osteoarthritis comprising:
(a) assaying for mRNA and/or protein levels of a substance selected from one or more of the following: α,β,γ, and δ isoforms of PI3K, IL-1, OSM, AGG-1, and COL-3 in a subject; and,
(b) administering to said subject with increased levels of α,β,γ, and δ isoforms of PI3K, IL-1, OSM, AGG-1, and COL-3 mRNA and/or protein levels compared to controls a PI3K modulator or PKB modulator in an amount sufficient to treat or ameliorate the pathological effects of osteoarthritis.

Another embodiment which contemplates and provides for additional diagnosis tools to detect subjects with OA relates to a diagnostic kit for detecting mRNA levels and/or protein levels of a substance selected from one or more of the following: α,β,γ, and δ isoforms of PI3K, IL-1, OSM, AGG-1, and COL-3 in a biological sample, said kit comprising:
(a) a polynucleotide of a substance selected from one or more of the following: α,β,γ, and δ isoforms of PI3K, IL-1, OSM, AGG-1, and COL-3 or a fragment thereof;
(b) a nucleotide sequence complementary to that of (a);
(c) a polypeptide of a substance selected from one or more of the following: α,β,γ, and δ isoforms of PI3K, IL-1, OSM, AGG-1, and COL-3, or a fragment thereof; wherein said polypeptide or fragment thereof or
(c) an antibody to a polypeptide of a substance selected from one or more of the following: α,β,γ, and δ isoforms of PI3K, IL-1, OSM, AGG-1, and COL-3
wherein components (a), (b), (c) or (d) may comprise a substantial component.

It will be appreciated that in any such kit, (a), (b), (c) or (d) may comprise a substantial component. It is also contemplated that said kit could comprise components (a)-(d) designed to detect levels of PI3K, PKB, IL-1, or OSM, including isoforms, related regulatory proteins or proteins modified by these proteins as discussed herein.

Factors for consideration for optimizing a therapy for a patient include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary for the treatment of osteoarthritis.

The pharmaceutical compositions disclosed herein are useful for treating, preventing and/or ameliorating OA, are to be administered to a patient at therapeutically effective doses to treat or ameliorate symptoms of such disorder. A therapeutically effective dose refers to that amount of the compound sufficient to result in either the inhibition of PI3K, PKB, IL-1, and/or OSM with resultant decrease in levels of AGG-1 and/or COL-3 or the prevention, treatment or amelioration of OA.

The inhibitory compositions of the invention can be administered as pharmaceutical compositions. Such pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or topical, oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. The route of delivery of the parenteral formulation can, for example, be intramuscular, intraperitoneal, subcutaneous, intravenous, or directly into an affected tissue of interest or into any other part of the body. In a preferred embodiment, the parenteral administration may take place via direct articular injection into an afflicted joint. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example, subcutaneous, intramuscular, intraperitoneal or articular) or by intramuscular or articular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose refers to that amount of active ingredient, for example, antisense oligonucleotides, triple helix DNA, ribozymes, RNA aptamer and double stranded RNA designed to inhibit PI3K, PKB, IL-1, OSM, AGG-1 or COL-3 gene expression, antibodies to these proteins or related regulatory proteins or fragments thereof, useful to treat, prevent and/or ameliorate the pathological effects of OA. Therapeutic efficacy may be determined by a PI3K or PKB enzyme assay or IL-1, OSM, AGG-1 or COL-3 expression level assay as herein described or by pharmaceutical procedures in cell cultures or experimental animals. For example, efficacy may be expressed as the level of inhibition of PI3K, PKB, IL-1, OSM, or resultant decrease in AGG-1 and/or COL-3. This efficacy may be expressed as an IC₅₀ (50% Inhibitory Concentration), where IC₅₀ is the quantity of a compound that must be added to an in an in vitro or in vivo PI3K, PKB, IL-1, or OSM, inhibition assay or AGG-1 or COL-3 expression assay to result in a level of PI3K or PKB enzyme activity or IL-1, OSM, AGG-1 or COL-3 expression levels which are a 50% reduction of the level of such enzymes or messenger RNA levels compared to those levels which occur when not inhibited under the same assay conditions. Another measure of efficacy is the ED₅₀ (50% Effective Dose) the ED₅₀ is the dose which is therapeutically effective in 50% of the population. Toxicity can be expressed as the LD₅₀ (the dose lethal to 50% of a population of test animals or cells). The dose ratio between LD₅₀ and therapeutic efficacy is the therapeutic index, and it can be expressed as the ratio, LD₅₀/ED₅₀. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

The following examples further illustrate the present invention, and are not intended to limit the invention.

### EXAMPLES

Methods:

The following techniques and experimental methods are used in Examples described herein. References in the Examples, which are made to the following techniques, are intended to identify experimental methods that are performed as described below.

Isolated Normal and Osteoarthritic Cartilage: Cartilage is isolated from the femoral condyles and the tibial plateaus of human knees. Normal cartilage is obtained from cadavers with no history of arthritis and without any macroscopic cartilage lesions. Osteoarthritic cartilage is obtained from patients undergoing joint replacement therapy.

Isolated Normal and Osteoarthritic Cartilage RNA: Normal and osteoarthritic cartilage is isolated from femoral condyles and the tibial plateaus of human knees as described above. The isolated cartilage is flash frozen in a liquid nitrogen bath.

RNA Isolation: RNA is isolated from flash frozen cartilage by homogenizing in a freezer mill and extracting the homogenate in 1 ml/100 mg tissue Trizol (Life Technologies, Rockville, Md.). The homogenized cartilage samples are extracted with chloroform, centrifuged at 15,000 times gravity for 20 minutes, and the aqueous phase is collected. An equal volume of 70% ethanol is added to the aqueous phase, mixed, and then applied to RNeasy columns (Qiagen, Valencia, Calif.). RNA concentrations are determined using RiboGreen reagent (Molecular Probes, Eugene, Oreg.).

cDNA from Isolated Normal and Osteoarthritic Cartilage: A quantity of 1 μg total Isolated Normal or Osteoarthritic Cartilage RNA is treated with DNase (Ambion, Austin, Tex.) in DNase buffer (Ambion, Austin, Tex. in a quantity sufficient to make 10 μl total reaction volume and heated at 37° C. for 30 minutes. The DNase enzyme is inactivated by adding EDTA and heating at 75° C. for 5 min. cDNA is then synthesized from the reaction mixture above using Oligo dT₁₂₋₁₈ with the Superscript Preamplification System (Life Technologies, Rockville, Md.). The final reaction volume is then adjusted to 300 μl. Cyclophilin RT-PCR is performed on each sample to allow comparative normalized PI3K, PKB, AGG-1, or COL-3 gene quantitation. Reverse transcriptase polymerase chain reaction (RT-PCR) for cyclophilin is first performed using 3 μl template utilizing the Expand Long PCR System (Roche Biochemicals, Indianapolis, Ind.). Cyclophilin RT-PCR is performed on each sample for 28 cycles to prevent saturation of the PCR products, whereas 35-40 cycles is necessary to detect less abundant genes. Loading dye is added to the 50 μl RT-PCR reaction mix, 10 μl of the dyed RT-PCR reaction mix is loaded onto a 4-20% TBE gradient gel (BioRad, Hercules, Calif.)) and separated at 200V for 45 min. The gel is stained in 1×TBE (Sigma, St. Louis, Mo.) solution containing SYBR green (1:10,000) (Molecular Probes, Eugene, Oreg.) for 1 hour and then scanned on the Storm 860—Blue Fluorescence/Chemifluorescence scanner (Molecular Dynamics, Division of Amersham Pharmacia, Piscataway, N.J.). Individual bands are quantitated using ImageQuant software (Molecular Dynamics, Division of Amersham Pharmacia, Piscataway, N.J.). The fluorescence value determined for each PCR band representing a particular gene is normalized against the fluorescence value for the cyclophilin band in the same donor sample.

Chondrocyte Culture: Isolated normal and osteoarthritic cartilage samples are rinsed in phosphate buffered saline (PBS) minced and digested with protease from Streptomyces griseus (Sigma, St. Louis) and collagenase-2 (Worthington Biochemicals, Lakewood, N.J.). The digestion step above liberates individual chondrocytes from the isolated cartilage samples. Chondrocytes are seeded at high density 1×10⁶ cells/well in 6 well plates in high glucose Dulbecco's Modified Eagle's Medium (DMEM) (Invitrogen Corporation, Carlsbad, Calif.) containing 10% heat inactivated bovine serum (Invitrogen Corporation, Carlsbad, Calif.) and maintained in a CO₂ incubator (Kendro Laboratory Products, Newtown, Conn.) at 5% CO₂ at 37° C. until cells reached 80% confluence

Induction or Inhibition of PI3KI3K/PKB In Cultured Chondrocytes: In all studies described below chondrocytes were cultured in DMEM medium with or without 10% Fetal Bovine Serum (Invitrogen Life Technologies, Carlsbad, Calif.). Cultured chondrocyte cells are incubated overnight in serum free medium (DMEM) and then treated, as one of the following types of samples, 1) control samples, by incubating the cultures for 24 hour in serum free medium (DMEM) 2) induced samples, by incubating the cultures for 24 hours in serum free medium containing the PI3K/PKB inducer PDGF-BB (Invitrogen—Life Technologies Carlsbad, Calif.) or 3) PI3K/PKB inhibited samples, by pre-incubating the samples for 1 hour with the PI3K/PKB inhibitor LY294002 (Sigma, St. Louis, Mo.) and then incubating the cultures for 24 hours in serum free medium (DMEM) containing the PDGF-BB. FIG. 3 depicts activation of PI3K/PKB expression by PDGF in primary human chondrocytes. FIG. 4 depicts LY249002 (2-(4-morpholinyl)-8-phenyl-4H-1-benzopyran-4-one) inhibition of PDGF activated PI3K/PKB expression in primary human chondrocytes.

Western Blotting—Cells are incubated in serum free medium (DMEM) overnight, and then incubated for 10 minutes in fresh serum free media containing PDGF-BB (50 ng/ml)+/−LY294002 (20 μM). Cells are washed in ice cold PBS, scraped and sonicated in ice cold lysis buffer containing 50 mM Tris pH 7.4, 1% NP-40, 0.25% Na deoxycholate, 150 mM NaCl, 1 mM EGTA, 1 mM PMSF, aprotinin, leupeptin, and pepstatin (all at 1 μg/ml 1 mM Na vanadate, 1 mM NaF, and 1 μM microcystin) (Sigma, St Louis, Mo.). The protein concentration of the lysate is determined using the micro bicinchoninic acid assay (Pierce, Rockford, Ill.) and 20 μg lysate is run per lane for phospho-PKB and phospho-forkhead and 5 μg for anti-His antibody [rabbit anti-phospho-PKB-Ser473 NEB, rabbit anti-phospho-FKHRL1-Thr32 NEB, mouse anti penta-His (Qiagen, Valencia, Calif.)]. Peroxidase labeled secondary antibodies are purchased from Jackson Laboratories (Bar, Harbor, Me.). Blots are visualized with ECL reagent (Amersham, Piscataway, N.J.). Western blots are run using the media from PDGF (Invitrogen-Life technologies, Carlsbad, Calif.) and/or LY294002 (Sigma, St. Louis, Mo.) 24 hour treated chondrocytes and probed with mouse anti-COL-3 (MMP-13, Oncogene, Cambridge, Mass.) and visualized with ECL (+) reagent (Amersham, Piscataway, N.J.).

Retroviral-PKB Construct—A retroviral construct is prepared from Myc-His tagged murine PKB-1 (Genbank accession no. NM-009652) which shares >95% homology to human PKB-1. This construct contains c-src derived residues required for myristoylation at the 5′ end (Upstate Biotechnologies, Lake Placid, N.Y.). The sequence is PCR amplified, sequence verified and inserted into a MoMuLV (Moloney murine leukemia virus) based retroviral expression vector. The plasmid is transiently transfected using calcium phophate into GP2-293 cells stably expressing the viral gag and pol proteins (Clontech, Palo Alto, Calif.). The packaging cells are also co-transfected with VSV-G coat protein (Stratagene, La Jolla, Calif.) In order to generate psudotyped retroviral particles. Viral supernatants are collected 48 hours later for transduction experiments. The retroviral vector alone is transfected as a control. Cells over-expressing PKB are transduced in two rounds as described below. After the second round of transduction, cells used to measure PKB and FKHRL1 proteins are allowed to recover overnight in serum free medium.

Retrovirus Transduced Chondrocytes: To maximize transfection efficiency chondrocytes are retrovirus transduced in 2 rounds. In order to accomplish this, Normal and osteoarthritic cartilage are isolated and digested with protease from Streptomyces griseus (Sigma, St. Louis) and collagenase-2 (Worthington Biochemicals, Lakewood, N.J.) as described above. However, the liberated chondrocytes are now seeded for culture at lower densities than described above to allow cell replication. Chondrocytes are seeded at 2×10⁵ cells per well in a 6-well plate, 24 hours prior to transduction in 8 μg/ml polybrene (Sigma, St. Louis, Mo.). Otherwise cells are seeded as described above for cultured chondrocytes. The following day the cells are transduced as follows. The media is removed and 3 ml fresh viral supernatant prepared as described above containing 10 μM HEPES and 8 μg/ml polybrene (Sigma, St. Louis, Mo.) is added to the cells. Cells are centrifuged in a swinging bucket rotor at 32° C., 1000×g, for 1.5 hours. Following centrifugation 3 ml fresh media containing polybrene is added to each well. The cells are further incubated at the same condiations as described above for 16 to 24 hours. The media is then replaced with fresh media without polybrene and cells are incubated for an additional 24 hours. Following this first transduction, a second round of transduction is performed followed by another 24 hour recovery period. Although cells are seeded at low density they still retained their chondrocytic nature assessed by their ability to produce nitrite in response to IL-1 as described below.

Nitrite Production Assay: Production of NO was estimated by measuring nitrite accumulation in chondrocyte culture medium using the Griess method as described by Green et al. (L.C. Green Analytical Biochemistry 126, 131-138 (1982)) which is incorporated herein by reference. Briefly, retrovirus transduced chondrocytes were treated with 2 ng/ml IL-1 β (Peprotech, Rocky Hill, N.J.). After 24 hours of incubation with IL-1, the culture media was collected and 50 ul of supernatant from each culture well was added to an equal volume of Greiss reagent (1:1, v/v, of 0.1% N-naphthylethylenediamine dihydrochloride in distilled water and 1% sulfanilamide in 5% phosphoric acid) in a 96-well flat-bottomed plate (Corning, Corning, N.Y.). Absorbance was measured at 550 nm using a Versemax microplate reader (Molecular Devices, SunnyVale, Calif.). An average of measurements from duplicate wells was used in the final analysis. Nitrite concentration was calculated from a standard curve prepared for each experiment using NaNO2 as the standard.

ELISA for COL-3—Chondrocyte culture cells are incubated in serum free media (DMEM) overnight, and then incubated for 1 hour in fresh serum free media without or with (50 ng/ml) PDGF-BB (Invitrogen-Life Technologies, Carlsbad, Calif.) is added to each well and incubated for an additional 24 hours. Cell culture supernatants are collected and the levels of COL-3 protein are determined for each treatment using Amersham (Piscataway, N.J.) human collagenase-3 ELISA. FIG. 5 depicts the ELISA of COL-3 protein secretion and inhibition by LY249002.

PCR Primers: Table 1 below lists the PCR primers that were used in the procedures described herein and above.

TABLE 1

| | |
|---|---|
| AGG-1-Primer set-1: | Sense-5′-CCCCCGGAATGGTGGCAAGTA-3′ |
| | Antisense-5′-GTGGGGGAGGGCATCAGCGTGTATTC-3′ |
| AGG-1-Primer set-2: | Sense-5′-GGTCGCTGCCTCCACATGGACCAGCTCCAGGACTT-3′ |
| | Antisense-5′ GGAGCCTGACTGCTTGCTGCAACCAGAACCGTCCC-3′ |
| Collagenase-3: | Sense-5′-CATTTGATGGGCCCTCTGGCCTGC-3′ |
| | Antisense-5′-TTTAGGGTTGGGGTCTTCATCTC-3′ |
| Cyclophilin: | Sense-5′-TGGCACAGGAGGAAAGAGCATC-3′ |
| | Antisense-5′-AAAGGGCTTCTCCACCTCGATC-3′ |
| P13K/p110α: | Sense-5′-GACTTATTGAGGTGGTG-3′ |
| | Antisense-5′ GGCATGCTGTCGAATAG-3′ |
| P13K/p110β: | Sense-5′-GCTAATGTGTCAAGTCG-3′ |
| | Antisense-5′-CCGATTACCAAGTGCTC-3′ |
| P13K/p110γ: | Sense-5′-CCTGCAGAATTCTCAAC-3′ |
| | Antisense-5′-CACAATCTCGATCATTC-3′ |
| P13K/p110δ: | Sense-5′-GTACTCCGTCAGACACC-3′ |
| | Antisense-5′-CATGATGTTGTCGCTGTG-3′ |

Statistical Analysis—Statistical analysis of RT-PCR data is performed by Students t-test.

### Example 1

Assay for PI3K Inhibitors which Affect AGG-1 and COL-3 Production

The levels of the matrix degrading enzymes AGG-1 and COL-3 are increased in osteoarthritic cartilage versus normal cartilage. These altered levels of AGG-1 and COL-3 proteases may contribute to the initiation and progression of osteoarthritis. A comparison of the AGG-1 and COL-3 gene expression levels in non-OA and OA chondrocytic lysate is shown in Tables 2 and 3 below. An inhibitor of proliferation of AGG-1 and COL-3 gene expression or protein production would be a potentially useful agent for treatment of OA. Therefore, this example describes a method for screening for compounds that have the ability to decrease AGG-1 and/or COL-3 gene or protein production through PI3K and/or PKB modulation. The inhibitors tested in this assay are examined for their effect on AGG-1 and/or COL-3 gene expression.

TABLE 2
Expression Of Collagenase-3 mRNA In Normal
And OA Cartilage (RT-PCR)

| Normal Cartilage | OA Cartilage |
|---|---|
| 7.38 | 63.90 |
| 7.68 | 31.41 |
| 7.04 | 40.71 |
| 7.55 | 20.23 |
| 6.79 | 161.27 |
| 7.05 | 192.41 |
| 6.45 | |
| 44.54 | |

Values are expressed as arbitrary fluorescent units. The fluorescent values for sample without reverse transcriptase and without template were below the level of detection.

TABLE 3
Expression Of Aggrecanase-1 mRNA In Normal
And OA Cartilage (RT-PCR)

| Normal Cartilage | OA Cartilage |
|---|---|
| 5.31 | 56.03 |
| 5.14 | 247.03 |
| 5.58 | 461.12 |
| 178.64 | 255.52 |
| 16.15 | 311.63 |
| 51.64 | 248.75 |
| 6.18 | |
| 370.20 | |

Values are expressed as arbitrary fluorescent units. The fluorescent values for sample without reverse transcriptase and without template were below the level of detection.

To generate a screening assay that would be useful for testing candidate compounds for their ability to treat and/or prevent osteoarthritis, via PI3K and/or PKB modulation or inhibition, we have created a screen using the PI3K inhibitor LY294002 as a control inhibitor. To screen compounds for their PI3K inhibitory activity, human chondrocytes may be obtained as described herein above from individuals diagnosed with active osteoarthritis and then cultured as described above. Cells may be incubated in serum free medium overnight, and then incubated for 1 hour, prior to addition of PDGF-BB (Invitrogen-Life Technologies, Carlsbad, Calif.), in either fresh serum free medium containing 50 ng/ml PDGF-BB (negative control) fresh serum free medium containing PDGF-BB (50 ng/ml) plus 20 μM LY294002 (Sigma, St. Louis, Mo.) (positive control), or fresh serum free medium containing 50 ng/ml PDGF-BB plus 10 or 20 uM test compound (test sample). The cells may then be washed and lysed as described above and the lysates probed for either AGG-1 or COL-3 mRNA levels via reverse transcription PCR and Western blot detection as decribed above. Those candidate compounds which lower the expressed levels of AGG-1 or COL-3 mRNA demonstrate inhibitory activity and may be useful to clinically treat, prevent or ameliorate OA and/or related conditions.

Results:

The induction of AGG-1 and COL-3 by PDGF is associated with and is dependent on activation of the PI3K/PKB pathway as shown in Tables 4, and 5 below. Collectively these data demonstrate that PI3K and/or PKB activate AGG-1 and COL-3 gene expression and protein secretion. The data also show that the PI3K inhibitor, LY2490002, decreases PDGF induced activation of PKB (Table 3) and also inhibits the downstream production of mRNA for AGG-1 and COL-3 (Table 4). The PI3K inhibitor LY2490002 also inhibits secretion of COL-3 protein as shown in Table 5. Collectively, these findings suggest that the PI3K enzyme is part of an important pathway leading to the induction of the cartilage matrix degrading enzymes AGG-1 and COL-3.

TABLE 4
Activation of PI3K/PKB pathway by PDGF in primary human
chondrocytes and blockade by the PI3K Inhibitor LY294002.
Chondrocyte Samples Probed with Anti Phospho-PKB

| | | |
|---|---|---|
| Untreated | 0 | 0 |
| PDGF (50 ng/ml) | 17940 | 22369 |
| PDGF (50 ng/ml) + LY294002 (20 μM) | 4706 | 3408 |

Chondrocyte Samples Probed with Anti-Phospho-FKHRL1

| | | |
|---|---|---|
| Untreated | 2134 | 1837 |
| PDGF (50 ng/ml) | 15838 | 16507 |

Depicts activation of PI3K/PKB by PDGF in primary human chondrocytes (non-OA joints) and resulting inhibition of activation by LY294002: western blots of chondrocyte lysates were probed with anti-phospho-PKB in top set of data.
In second set of data chondrocyte lysate samples were probed with anti-phospho-FKHRL1 (downstream promoter of PKB) to positively identify that PKB pathway was stimulated.

TABLE 5
PDGF induces COL-3 and AGG-1 Gene Expression

| | | | PDGF (50 ng/ml) + |
|---|---|---|---|
| Gene | Untreated | PDGF(50 ng/ml) | LY294002 (20 μM) |
| Aggrecanase-1 | 0 | 418 | 0 |
| Collagenase-3 | 5 | 1242 | 302 |

Depicts LY294002 inhibition of PDGF stimulated induction of AGG-1 and COL-3 mRNA. PCR amplicons of AGG-1 and COL-3 from primary human chondrocyte lysates (non-OA joints) were analyzed by agarose gel electrophoresis. Gel images were analyzed by scanning them into the Stratagene Eagle Eye II still video system (Stratagene, La Jolla, CA).

### Example 2

Rapid Screening Method of PI3K Inhibitors for Resultant Effects on COL-3

Inhibitors which are discovered in the Assay for PI3K inhibitors which affect AGG-1 and COL-3 Production (Example 1) above can be secondarily screened for effect on COL-3 protein secretion using the ELISA assay as described below. While this assay, as described, is commercially available, it would be known to those skilled in the art how to create a de novo ELISA based immuno-assay utilizing radiolabelled monoclonal antibodies to human COL-3 protein. This commercially based screening method is commercially available from Amersham (Piscataway, N.J.).

ELISA for COL-3:

Chondrocyte cells isolated as described above are incubated in serum free media overnight, and then incubated for 1 hour in fresh serum free medium (DMEM) with or without LY294002. Then 50 ng/ml PDGF-BB (Sigma, St. Louis, Mo.) is added to each well and incubated for an additional 24 hours. Chondrocyte cell culture supernatants are collected and the levels of COL-3 protein are determined for each treatment using the commercially available Amersham (Piscataway, N.J.) human collagenase-3 ELISA based assay.

Results:

The effect of the control standard PI3K inhibitor, LY294002, on the levels of COL-3 protein secretion in primary human chondrocytes is shown in table 6 below. These data were generated using the ELISA test as hereinabove described.

TABLE 6
LY294002 Inhibits Collagenase-3 Protein Secretion Mediated By PDGF

| | Collagenase-3 |
|---|---|
| Sample | (pg/ml) |
| Control | 2491 ± 147 |
| 1 μM LY 294002 | 2374 ± 58 |
| 5 μM LY 294002 | 1555 ± 104 |
| 20 μM LY 294002 | 342 ± 8 |

Primary human chondrocytes (non-OA joints) were treated with various concentrations of the PI3-Kinase inhibitor LY294002 in the presence of PDGF (50 ng/ml) for 24 hr. Collagenase-3 protein levels in the chondrocyte supernatants were measured using human Collagenase-3 ELISA system (Sigma, St. Louis, MO).

### Example 3

Detection of Altered Levels of PI3K Isoforms as a Diagnostic Tool:

This method provides a diagnostic tool by which one can detect altered levels of PI3K isoforms, α,β,γ, and δ. The applicants have discovered that surprisingly there are altered levels of these PI3K isoforms in OA afflicted joint chondrocytes as compared to non-OA joints. Thus, this method offers a way to diagnose those subjects who are suspected of having OA.

Chondrocyte cells may be isolated from joints in subjects suspected of being afflicted with OA. The cells may be placed in serum free medium (DMEM) The cells may then be washed and lysed as described above in METHODS and the lysates probed for PI3 Kα, PI3Kβ, PI3Kγ, and/or PI3Kδ mRNA levels via reverse transcription PCR and Western blot detection as decribed above. Those subjects who display altered PI3K isoform levels or trends compared to Normal Cartilage samples, as shown in Table 7 below, may be diagnosed for OA. For example, if a subject displays the following trends compared to the control Normal Cartilage samples shown in Tables 7, 8, 9, and 10 below that subject may be positively identified as having OA:
Decreased PI3Kα levels; and/or
Decreased PI3Kβ levels; and/or
Increased PI3Kγ levels; and/or
Decreased PI3KB levels.

Those subjects that display one or more of the above described trends in PI3K isoform levels may be positvely diagnosed for the existence of OA and thus identified as potentially responsive to treatment with a PI3K inhibitor to reduce levels of AGG-1 and or COL-3.

TABLE 7
Expression Of PI-3 Kinase Alpha mRNA In
Normal And OA Cartilage (RT-PCR)

| Normal Cartilage | OA Cartilage |
|---|---|
| 467.60 | 103.99 |
| 281.22 | 71.57 |
| 206.40 | 107.34 |
| 192.60 | 167.16 |
| 101.50 | 87.20 |
| 57.90 | 38.55 |
| 176.53 | |
| 120.91 | |

Values are expressed as arbitrary florescent units. The florescent values for sample without reverse transcriptase and without template were below the level of detection.

TABLE 8
Expression Of PI-3 Kinase Beta mRNA In
Normal And OA Cartilage (RT-PCR)

| Normal Cartilage | OA Cartilage |
|---|---|
| 1761.09 | 12.16 |
| 991.03 | 8.10 |
| 659.12 | 12.50 |
| 752.52 | 18.35 |
| 387.76 | 10.15 |
| 327.09 | 6.71 |
| 1164.01 | 7.32 |
| 27.41 | |

Values are expressed as arbitrary florescent units. The florescent values for sample without reverse transcriptase and without template were below the level of detection.

TABLE 9
Expression Of PI-3 Kinase Gamma mRNA In
Normal and OA Cartilage (RT-PCR)

| Normal Cartilage | OA Cartilage |
|---|---|
| 16.60 | 45.92 |
| 7.55 | 29.08 |
| 5.11 | 26.57 |
| 74.60 | 55.17 |
| 2.06 | 88.85 |
| 48.76 | 30.76 |
| 5.47 | |
| 129.17 | |

Values are expressed as arbitrary florescent units. The florescent values for sample without reverse transcriptase and without template were below the level of detection.

TABLE 10
Expression Of PI-3 Kinase Delta mRNA In
Normal And OA Cartilage (RT-PCR)

| Normal Cartilage | OA Cartilage |
|---|---|
| 204.94 | 1.90 |
| 90.53 | 15.46 |
| 5.84 | 2.02 |
| 64.45 | 4.66 |
| 37.71 | 3.58 |
| 24.85 | 4.40 |
| 74.63 | |
| 4.37 | |

Values are expressed as arbitrary florescent units. The florescent values for sample without reverse transcriptase and without template were below the level of detection.

### Example 3

Superinduction of AGG-1 and COL-3 by IL-1 and OSM via PI3K/PKB Pathway

We show that IL-1 and OSM synergistically cause super-induction of AGG-1 and COL-3 (MMP-13) and this effect is also blocked by inhibiting the PI3 kinase pathway with either LY294002 or with an retroviral vector for a dominant negative form of PKB (Akt).

Cytokines such as IL-1 and OSM have been demonstrated to have role in cartilage loss associated with OA. They have been shown to significantly up-regulate matrix metalloproteinase gene expression. We demonstrate using the PI3 kinase inhibitor LY294002 and over-expression of the dominant mutant of Akt/PKB, for the first time that the PI3 kinase pathway has a significant role in the cytokine mediated induction of Aggrecanase-1 and Collagenase-3 in human articular chondrocytes.

Methods:

Chondrocytes were isolated from 2 normal and 2 OA patents. Cells alone or those over expressing the dominant negative form of Akt (retroviral mediated gene transfer) were treated with IL-1 beta, OSM or IL-1 beta+OSM with and without the PI3K inhibitor LY294002. RNA was isolated and the levels of Agg-1, Col-3 mRNA were measured by real time PCR. All treatments in four different donors were done in duplicate. Values obtained from a typical experiment are shown in the table.

RNA Isolation and Real Time PCR:

Total RNA was isolated from pooled duplicate 96-well plates using Qiagen RNeasy 96 kit (Qiagen, Valencia, Calif.) according to the manufacturer's instructions. The optional on-column Dnase I digestion was employed to eliminate contaminating genomic DNA. First strand cDNA was synthesized using random primers with the High-Capacity cDNA Archive kit (PE Applied Biosystems, Foster City, Calif.) in a 100 ul reaction volume.

Real time PCR was performed in a 384-well format on the ABI Prism 7900HT Sequence Detection System (Applied Biosystems, Foster City, Calif.) using SYBR Green PCR Master Mix (Applied Biosystems, Foster City, Calif.). The 20 ul reaction contained 5 ul cDNA, 200 nM forward and reverse primers, and SYBR Green PCR Master Mix. The default cycling program (95° C. for 10′ and 40 cycles of 95° C., 15″, 60° C., 1′) was followed by a slow ramping step by which a melting curve is generated to confirm the specificity of the PCR product and the absence of primer dimers.

Primers:

For Example 4, primers for cDNA targets were designed with Primer Express software (Applied Biosystems, Foster City, Calif.) under default parameters and reaction conditions and are as follows:

Aggrecanase I forward
5′TTTCCCTGGCAAGGACTATGA3′
Aggrecanase I reverse
5′AATGGCGTGAGTCGGGC3′
MMP13 forward
5′TGATCTCTTTTGGAATTAAGGAGCAT3′
MMP13 reverse
5′ATGGGCATCTCCTCCATAATTTG3′
GAPDH forward
5′ATGGGGAAGGTGAAGGTCG3′
GAPDH reverse
5′TAAAAGCAGCCCTGGTGACC3′

Amplification of GAPDH was used to standardize the amount of sample cDNA added to the reaction. Changes in gene expression were calculated using the Comparative Ct method which makes use of a calibrator sample. This is the sample with which all others will be compared and whose value is therefore set to 1. All other values are therefore expressed relative to the calibrator. The retroviral vector containing no cDNA insert represents the calibrator sample. The amount of target, relative to the calibrator is calculated according to the formula 2^{□□CT} as outlined in ABI User Bulletin #2 (PE Applied Biosystems, Foster City, Calif.).

Results:

Table 11 shows that IL-1 and OSM synergistically cause super-induction of AGG-1 and COL-3 (MMP-13) and this effect is also blocked by inhibiting the PI3 kinase pathway with either LY294002 or with an retroviral vector for a dominant negative form of PKB (Akt).

TABLE 11

| | Fold Induction of mRNA | |
|---|---|---|
| Cell Treatment | Aggrecanase-1 | Collagenase-3 |
| Untreated | 2 | 1 |
| IL-1 | 180 | 150 |
| AKT Dominant Negative + IL-1 | 38 | 29 |
| LY294002 + IL-1 | 22 | 5 |
| Oncostatin M | 221 | 63 |
| Akt Dominant Negative + Oncostatin M | 40 | 13 |
| LY294002 + Oncostatin M | 45 | 10 |
| IL-1 + Oncostatin M | 339 | 123 |
| Akt Dominant Negative + IL- | 42 | 13 |
| 1 + Oncostatin M | | |
| LY 294002 + IL-1 + Oncostatin M | 49 | 19 |
| PDGF | 10 | 36 |
| Akt dominant Negative + PDGF | 1 | 3 |
| LY 294002 + PDGF | 2 | 2 |

This is the representative result from chondrocytes isolated from one OA patient. Essentially similar results were obtained from 4 other patients.

We claim:

1. A method to prevent, treat, or ameliorate a disease associated with or caused by altered levels of Aggrecanase-1 or Collagenase-3 comprising administering to a subject in need thereof an effective amount of a compound capable of modulating Phospho-Inositide 3 Kinase, Protein Kinase B, IL-1, or OSM and through said modulation alter gene and/or protein expression of Aggrecanase-1 or Collagenase-3.

2. A method to prevent, treat, or ameliorate osteoarthritis comprising administering to a subject in need thereof an effective amount of a compound capable of modulating Phospho-Inositide 3 Kinase, Protein Kinase B, IL-1, or OSM, and through said modulation alter gene and/or protein expression of Aggrecanase-1 or Collagenase-3.

3. The method of claim 2 wherein said Phospho-Inositide 3 Kinase modulator, Protein Kinase B modulator, IL-1 modulator, or OSM modulator inhibits in said subject the enzyme activity of one or more proteins selected from the following: Phospho-Inositide 3 Kinase and Protein Kinase B.

4. The method of claim 2 wherein said Phospho-Inositide 3 Kinase modulator or Protein Kinase B modulator inhibits in said subject gene or protein expression of one or more proteins selected from the following: Phospho-Inositide 3 Kinase, Protein Kinase B, IL-1, OSM, Aggrecanase-1, and Collagenase-3.

5. The method of claim 2 wherein said Phospho-Inositide 3 Kinase modulator or Protein Kinase B modulator is LY249002 2-(4-morpholinyl)-8-phenyl-4H-1-benzopyran-4-one) in free or pharmaceutically acceptable salt forms.

6. The method of claim 2 wherein said Phospho-Inositide 3 Kinase modulator, Protein Kinase B modulator, IL-1 modulator, or OSM modulator comprises any one or more substances selected from the following: antisense oligonucleotides, triple helix DNA, single stranded DNA, ribozymes, RNA aptamer and double stranded RNA wherein said substances are designed to inhibit gene expression of PI3K, PKB, IL-1, OSM, AGG-1 or COL-3 proteins.

7. The method of claim 2 wherein said Phospho-Inositide 3 Kinase modulator, Protein Kinase B modulator, IL-1 modulator, or OSM modulator comprises one or more antibodies to Phospho-Inositide 3 Kinase, Protein Kinase B, IL-1, or OSM, or fragments thereof, wherein said antibodies or fragments thereof can inhibit the enzyme activity of a protein selected from the following: Phospho-Inositide 3 Kinase and Protein Kinase B.

8. A method to prevent, treat, or ameliorate osteoarthritis comprising administering to a subject in need thereof a pharmaceutical composition comprising an effective amount of a modulator of Phospho-Inositide 3 Kinase, Protein Kinase B, IL-1, OSM and through said modulation alter gene and/or protein expression of Aggrecanase-1 or Collagenase-3.

9. The method of claim 8 wherein said Phospho-Inositide 3 Kinase modulator, Protein Kinase B modulator, IL-1 modulator, or OSM modulator inhibits in said subject the enzyme activity of one or more proteins selected from the following: Phospho-Inositide 3 Kinase and Protein Kinase B.

10. The method of claim 8 wherein said Phospho-Inositide 3 Kinase modulator or Protein Kinase B modulator inhibits in said subject gene or protein expression of one or more proteins selected from the following: Phospho-Inositide 3 Kinase, Protein Kinase B, IL-1, OSM, Aggrecanase-1, and Collagenase-3.

11. The method of claim 8 wherein said Phospho-Inositide 3 Kinase modulator or Protein Kinase B modulator is LY249002 (2-(4-morpholinyl-8-phenyl-4H-1-benzopyran-4-one) in free or pharmaceutically acceptable salt forms.

12. The method of claim 8 wherein said Phospho-Inositide 3 Kinase modulator, Protein Kinase B modulator, IL-1 modulator, or OSM modualtor comprises one or more antibodies to Phospho-Inositide 3 Kinase, Protein Kinase B, IL-1 modulator, or OSM modulator, or fragments thereof, wherein said antibodies or fragments thereof can inhibit the enzyme activity of a protein selected from the following: Phospho-Inositide 3 Kinase and Protein Kinase B.

13. A method to identify modulators useful to prevent, treat, or ameliorate osteoarthritis comprising assaying for the ability of a candidate modulator to inhibit Phospho-Inositide 3 Kinase, Protein Kinase B, IL1, or OSM, and through said inhibition alter gene and/or protein expression of Aggrecanase-1 or Collagenase-3.

14. The method of claim 13 wherein said method further comprises assaying for the ability of an identified Phospho-Inositide 3 Kinase, Protein Kinase B, IL-1, or OSM, inhibitory modulator to reverse the pathological effects observed in animal models of osteoarthritis.

15. The method of claim 13 wherein said modulator inhibits the enzyme activity of one or more proteins selected from the following: Phospho-Inositide 3 Kinase and Protein Kinase B.

16. The method of claim 13 wherein said modulator inhibits gene or protein expression of one or more proteins selected from the following: Phospho-Inositide 3 Kinase, Protein Kinase B, IL-1, OSM, Aggrecanase-1, and Collagenase-3

17. The method according to claim 13 wherein said method further comprises assaying for the ability of an identified inhibitory modulator to reverse the pathological effects observed in clinical studies with subjects with osteoarthritis.

18. The method of claim 13 wherein said Phospho-Inositide 3 Kinase modulator, Protein Kinase B modulator, IL-1 modulator, or OSM modulator comprises any one or more substances selected from the following: antisense oligonucleotides, triple helix DNA, single stranded DNA, ribozymes, RNA aptamer and double stranded RNA wherein said substances are designed to inhibit gene expression of PI3K, PKB, IL-1, OSM, AGG-1 or COL-3 proteins.

19. The method of claim 13 wherein said Phospho-Inositide 3 Kinase modulator, Protein Kinase B modulator, IL-1 modulator, or OSM modulator comprises one or more antibodies to Phospho-Inositide 3 Kinase, Protein Kinase B, IL-1, OSM, or fragments thereof, wherein said antibodies or fragments thereof can inhibit the enzyme activity of a protein selected from the following: Phospho-Inositide 3 Kinase and Protein Kinase B

20. A pharmaceutical composition comprising a Phospho-Inositide 3 Kinase modulator, Protein Kinase B modulator, IL-1 modulator, or OSM modulator in an amount effective to prevent, treat, or ameliorate osteoarthritis in a subject in need thereof.

21. The pharmaceutical composition according to claim 20 wherein said modulator inhibits the enzyme activity of one or more proteins selected from the following: Phospho-Inositide 3 Kinase and Protein Kinase B.

22. The pharmaceutical composition according to claim 20 wherein said modulator inhibits gene or protein expression of one or more proteins selected from the following: Phospho-Inositide 3 Kinase, Protein Kinase B, IL-1, OSM, Aggrecanase-1, and Collagenase-3.

23. The pharmaceutical composition of claim 20 wherein said modulator is LY249002 (2-(4-morpholinyl)-8-phenyl-4H-1-benzopyran-4-one) in free or pharmaceutically acceptable salt forms.

24. The pharmaceutical composition of claim 20 wherein said modulator comprises any one or more substances selected from the following: antisense oligonucleotides, triple helix DNA, single stranded DNA, ribozymes, RNA aptamer and double stranded RNA wherein said substances are designed to inhibit gene expression of PI3K, PKB, IL-1, OSM, AGG-1 or COL-3 proteins.

25. The pharmaceutical composition of claim 20 wherein said modulator comprises one or more antibodies to Phospho-Inositide 3 Kinase, Protein Kinase B, IL-1, OSM, or fragments thereof, wherein said antibodies or fragments thereof can inhibit the enzyme activity of a protein selected from the following: Phospho-Inositide 3 Kinase and Protein Kinase B.

26. A method to diagnose subjects affected with active or nascent osteoarthritis who may be suitable candidates for treatment with Phospho-Inositide 3 Kinase, Protein Kinase B modulators, IL-1 modulators, or OSM modulators comprising assaying mRNA levels of a substance selected from one or more of the following: α,β,γ, and δ isoforms of Phospho-Inositide 3 Kinase, IL-1, OSM, Aggrecanase-1, and Collagenase-3 in a biological sample from said subject wherein subjects with altered levels of said Phospho-Inositide 3 Kinase, IL-1, OSM, Aggrecanase-1, or Collagenase-3 compared to non-osteoarthritic control levels would be suitable candidates for Phospho-Inositide 3 Kinase modulator, Protein Kinase B modulator, IL-1 modulator, or OSM modulator treatment.

27. A method to prevent, treat, or ameliorate osteoarthritis comprising:
(a) assaying for mRNA and/or protein levels of a substance selected from one or more of the following: α,β,γ, and δ isoforms of Phospho-Inositide 3 Kinase, IL-1, OSM, Aggrecanase-1, and Collagenase-3 in a subject; and,
(b) administering to said subject with altered levels of α,β,γ, and δ isoforms of Phospho-Inositide 3 Kinase, IL-1, OSM, Aggrecanase-1, and Collagenase-3 mRNA and/or protein levels, compared to controls, a Phospho-Inositide 3 Kinase modulator, Protein Kinase B modulator, IL-1 modulator, or OSM modulator in an amount sufficient to treat or ameliorate the pathological effects of osteoarthritis.

28. A method to diagnose subjects affected with active or nascent osteoarthritis who may be suitable candidates for treatment with Phospho-Inositide 3 Kinase modulators, Protein Kinase B modulators, IL-1 modulators, or OSM modulators comprising assaying mRNA levels of a substance selected from one or more of the following: α,β,γ, and δ isoforms of Phospho-Inositide 3 Kinase, IL-1, OSM, Aggrecanase-1, and Collagenase-3 in a biological sample from said subject wherein subjects with increased levels of Phospho-Inositide 3 Kinase, IL-1, OSM, Aggrecanase-1, or Collagenase-3, compared to non-osteoarthritic control levels, would be suitable candidates for Phospho-Inositide 3 Kinase modulator, Protein Kinase B modulator, IL-1 modulator, or OSM modulator treatment.

29. A method to prevent, treat, or ameliorate osteoarthritis comprising:
(a) assaying for mRNA and/or protein levels of a substance selected from one or more of the following: α,β,γ, and δ isoforms of Phospho-Inositide 3 Kinase, IL-1, OSM, Aggrecanase-1, and Collagenase-3 in a subject; and,
(b) administering to said subject with increased levels of α,β,γ, and δ isoforms of Phospho-Inositide 3 Kinase, IL-1, OSM, Aggrecanase-1, and Collagenase-3 mRNA and/or protein levels, compared to controls, a Phospho-Inositide 3 Kinase modulator, Protein Kinase B modulator, IL-1 modulator, or OSM modulator in an amount sufficient to treat or ameliorate the pathological effects of osteoarthritis.

30. A diagnostic kit for detecting mRNA levels and/or protein levels of a substance selected from one or more of the following: α,β,γ, and δ isoforms of Phospho-Inositide 3 Kinase, IL-1, OSM, Aggrecanase-1, and Collagenase-3 in a biological sample, said kit comprising:
(a) a polynucleotide of a substance selected from one or more of the following: α,β,γ and δ isoforms of Phospho-Inositide 3 Kinase, IL-1, OSM, Aggrecanase-1, and Collagenase-3 or a fragment thereof;
(b) a nucleotide sequence complementary to that of (a);
(c) a polypeptide of a substance selected from one or more of the following: α,β,γ, and δ isoforms of Phospho-Inositide 3 Kinase, IL-1, OSM, Aggrecanase-1, and Collagenase-3, or a fragment thereof; wherein said polypeptide or fragment thereof or
(c) an antibody to a polypeptide of a substance selected from one or more of the following: α,β,γ, and δ isoforms of Phospho-Inositide 3 Kinase, IL-1, OSM, Aggrecanase-1, and Collagenase-3
wherein components (a), (b), (c) or (d) may comprise a substantial component.

31. Use of a compound capable of modulating Phospho-Inositide 3 Kinase Protein Kinase B, IL-1, or OSM for the manufacture of a medicament for the treatment of a disease associated with or caused by altered levels of Aggrecanase-1 or Collagenase-3.

32. Use of a compound capable of modulating Phospho-Inositide 3 Kinase Protein Kinase B, IL-1, or OSM for the manufacture of a medicament for the treatment osteoarthritis.

33. Use of 2-(4-morpholinyl)-8-phenyl-4H-1-benzopyran-4-one) for the manufacture of a medicament for the treatment of a disease associated with or caused by altered levels of Aggrecanase-1 or Collagenase-3.

34. Use of 2-(4-morpholinyl)-8-phenyl-4H-1-benzopyran-4-one) for the manufacture of a medicament for the treatment of osteoarthritis.
